(19) United States
(12) Patent Application Publication
(10) Pub. No.: US 2025/0017841 A1
(43) Pub. Date: Jan. 16, 2025

(54) COMPOUND CONTAINING ASCORBIC ACID DERIVATIVE

(71) Applicant: DR'S CHOICE CO., LTD., Tokyo (JP)

(72) Inventors: Akio YAMAMOTO, Chiyoda-ku (JP); Masaru NAKAMURA, Chiyoda-ku (JP); Masahiko NAKANO, Chiyoda-ku (JP); Kiyofumi MATSUMOTO, Chiyoda-ku (JP); Miki OTSUKA, Chiyoda-ku (JP); Takashi MIYAHARA, Chiyoda-ku (JP)

(73) Assignee: DR'S CHOICE CO., LTD., Tokyo (JP)

(21) Appl. No.: 18/706,232

(22) PCT Filed: May 27, 2022
(86) PCT No.: PCT/JP2022/021724

§ 371 Date: Apr. 30, 2024

(30) Foreign Application Priority Data
Nov. 7, 2021 (JP) 2021-181546

(51) Int. Cl. A61K 8/67; A61Q 19/02

(52) U.S. Cl. CPC A61K 8/676; A61Q 19/02; A61K 2800/522; A61K 2800/591

(57) ABSTRACT

The present invention relates to a browning-suppressed composition containing ascorbic acid 2-glucoside basic amino acid salt. Blending of the ascorbic acid 2-glucoside basic amino acid salt makes it possible to suppress browning of the composition.

### TECHNICAL FIELD

The present invention relates to a browning-suppressed composition containing ascorbic acid 2-glucosides.

### BACKGROUND ART

Ascorbic acid acts as a reducing agent, a collagen synthesis promoter, and an antioxidant, and in particular, the excellent antioxidant effect of ascorbic acid is known as a component that suppresses the generation of melanin in the skin and suppresses pigmentation such as pigmented macules and freckles, and is widely blended, for example, in medicines, foods, and cosmetics.

However, the ascorbic acid is unstable in a hygroscopic state or alkali in a bulk powder, and an aqueous solution of ascorbic acid is not only strongly acidic and irritating, but also easily oxidized, and typical amines involved in decomposition causes a Maillard reaction to cause browning, and browning reaction proceeds. As a result, a product containing a cosmetic including the ascorbic acid and moisture is likely to generate an odor due to browning, so that the value of the product is lost, and the amount added to the product is limited, and the impact on effectiveness is unavoidable (Non-Patent Literature 1).

From such a viewpoint, techniques of blending flavonoid glycoside with the ascorbic acid in order to suppress browning of ascorbic acid have been reported (Patent Literatures 1 and 2). It has also been reported that ascorbic acid 2-glucoside is used instead of the ascorbic acid.

However, although an aqueous solution of the ascorbic acid 2-glucoside has a low pH and slightly improved stability, it still has a drawback of browning. Therefore, a technique of blending the ascorbic acid 2-glucoside with a strong base substance, lecithin, an ether compound, an ester compound, and citric acid, for example, have also been reported (Patent Literatures 3 to 6).
Patent Literature 1: JP H04-099771 A
Patent Literature 2: JP H04-099730 A
Patent Literature 3: JP 2001-199863 A
Patent Literature 4: JP 2002-265344 A
Patent Literature 5: JP 2005-220029 A
Patent Literature 6: JP 2012-246248 A

### Non Patent Literature

Non Patent Literature 1: Journal for the Integrated Study of Dietary Habits, Vol. 26, No. 1, 7-10 (2015)

### SUMMARY OF INVENTION

### Technical Problem

However, browning reaction of ascorbic acid 2-glucoside could not be sufficiently suppressed by any means, and the browning could not be sufficiently suppressed while maintaining the excellent action and effect of a composition such as a cosmetic containing the ascorbic acid 2-glucoside.

Therefore, an object of the present invention is to provide a new means for suppressing browning of ascorbic acid 2-glucoside, and a browning-suppressed composition containing the ascorbic acid 2-glucoside.

### Solution to Problem

Therefore, the present inventors variously studied a new means for suppressing browning of ascorbic acid 2-glucoside. First, the present inventors focused on the fact that ascorbic acid is sugar acid having unsaturated lactone. From the viewpoint that products containing sugar and amino acid are likely to cause a Maillard reaction, so-called browning, at room temperature or by heating, the present inventors mixed various amino acids, amino acid/sugar mixtures, amino acid/amino acid ester mixtures, peptides, 2-amino-2-methyl-1-propanol, ¿-aminocaproic acid, and γ-aminobutyric acid, for example, with the ascorbic acid, and it was found that addition of amino acids to the ascorbic acid causes browning reaction to proceed rapidly.

Therefore, when a mixed system of ascorbic acid 2-glucoside and an amino acid was used as a test system for browning reaction of ascorbic acid 2-glucoside, and a component for suppressing the browning of ascorbic acid 2-glucoside was screened, it was found that, quite unexpectedly, the browning reaction can be remarkably inhibited by using ascorbic acid 2-glucoside basic amino acid salt instead of ascorbic acid 2-glucoside. The present inventors also found that the ascorbic acid 2-glucoside basic amino acid salt can be obtained as a powder having good storage stability, and an aqueous solution of the ascorbic acid 2-glucoside basic amino acid salt has slightly acidic pH to neutral pH, has little skin irritation, and is excellent as a component to be blended in various compositions such as cosmetics. Furthermore, the present inventors also found that when the ascorbic acid 2-glucoside basic amino acid salt and ascorbic acid or salt thereof are used in combination, a composition having good usability is obtained.

The present invention provides the following inventions [1] to [10].

[1] A browning-suppressed composition comprising ascorbic acid 2-glucoside basic amino acid salt.

[2] The composition according to [1], wherein the ascorbic acid 2-glucoside basic amino acid salt is one or more selected from the group consisting of ascorbic acid 2-glucoside lysine salt, ascorbic acid 2-glucoside arginine salt, ascorbic acid 2-glucoside histidine salt, and ascorbic acid 2-glucoside tryptophan salt.

[3] The composition according to [1] or [2], wherein pH of the composition is 5.0 or more and 7.5 or less.

[4] The composition according to any one of [1] to [3], which is a food composition, a pharmaceutical composition, or a cosmetic composition.

[5] A composition comprising (a) ascorbic acid 2-glucoside basic amino acid salt and (b) ascorbic acid or salt thereof.

[6] The composition according to [5], wherein (b) the ascorbic acid or the salt thereof is one or more selected from the group consisting of ascorbic acid, ascorbic acid alkali metal salt, ascorbic acid amino acid salt, and ascorbic acid amine salt.

[7] The composition according to [5] or [6], wherein (a) the ascorbic acid 2-glucoside basic amino acid salt is one or more selected from the group consisting of ascorbic acid 2-glucoside lysine salt, ascorbic acid 2-glucoside arginine salt, ascorbic acid 2-glucoside histidine salt, and ascorbic acid 2-glucoside tryptophan salt.

[8] The composition according to any one of [5] to [7], wherein pH of the composition is 5.0 or more and 7.5 or less.

[9] The composition according to any one of [5] to [8], which is a food composition, a pharmaceutical composition, or a cosmetic composition.

A powder of ascorbic acid 2-glucoside basic amino acid salt.

### Advantageous Effects of Invention

Since browning of an aqueous solution of ascorbic acid 2-glucoside basic amino acid salt is significantly suppressed and the aqueous solution has a weakly acidic to neutral pH, the aqueous solution has a whitening effect due to suppression of melanin generation and a wrinkle preventing effect due to activation of collagen generation, which are various functions of ascorbic acids, and is useful as a safe blending component of foods, medicines, or cosmetics without skin irritation.

In addition, since the ascorbic acid 2-glucoside basic amino acid salt can be obtained as a powder according to the present invention, the component can be stably stored for a long period of time, and can be blended in compositions such as cosmetics, foods, or medicines at a high concentration.

When the ascorbic acid 2-glucoside basic amino acid salt and ascorbic acid or salt thereof are contained, a food composition, a pharmaceutical composition, and a cosmetic composition which are excellent in storage stability and have good usability are obtained.

### BRIEF DESCRIPTION OF DRAWINGS

FIG. 1 is a diagram showing the degree of browning of L-ascorbic acid caused by addition of an amino acid.

FIG. 2 is a diagram showing an FT/IR spectrum of a powder of ascorbic acid 2-glucoside arginine salt.

FIG. 3 is a diagram showing the results of measuring absorption spectrum (wavelength: 480 nm) and evaluating degree of browning (chromaticity) of test solution collected every designated month (Example 1).

FIG. 4 is a diagram showing the results of measuring absorption spectrum (wavelength: 480 nm) and evaluating degree of browning (chromaticity) of test solution collected every designated month (Example 2).

### DETAILED DESCRIPTION OF THE INVENTION

The present inventors found that browning is suppressed in various compositions in which ascorbic acid 2-glucoside basic amino acid salt is blended.

Therefore, one aspect of the present invention is a browning-suppressed composition containing the ascorbic acid 2-glucoside basic amino acid salt.

The ascorbic acid in the present invention is a compound having IUPAC name of (R)-3, 4-dihydroxy-5-((S)-1,2-dihydroxyethyl) furan-2 (5H)-one, which is also referred to as vitamin C or L-ascorbic acid. The ascorbic acid acts as a reducing agent, a collagen synthesis promoter, and an antioxidant as described above, and in particular, the excellent antioxidant effect of ascorbic acid is known as a component that suppresses the generation of melanin in the skin and prevents pigmentation such as pigmented macules and freckles, and is widely used in medicines, foods, and cosmetics.

The ascorbic acid 2-glucoside is a substance in which glucose is bonded to carbon atom at the 2-position of ascorbic acid, and is metabolized in vivo to become ascorbic acid, and thus is regarded as provitamin of ascorbic acid. The ascorbic acid 2-glucoside basic amino acid salt used in the present invention is basic amino acid salt of the ascorbic acid 2-glucoside.

Here, examples of the ascorbic acid 2-glucoside basic amino acid include one or more selected from ascorbic acid 2-glucoside lysine salt, ascorbic acid 2-glucoside arginine salt, ascorbic acid 2-glucoside histidine salt, and ascorbic acid 2-glucoside tryptophan salt. Among them, the ascorbic acid 2-glucoside lysine salt and the ascorbic acid 2-glucoside arginine salt are more preferable.

The constituent molar ratio of the ascorbic acid 2-glucoside and basic amino acid in the basic amino acid salt of ascorbic acid 2-glucoside is preferably from 1:1.5 to 1.5:1, and more preferably from 1:1.2 to 1.2:1.

The ascorbic acid 2-glucoside basic amino acid salt can be produced by reacting the basic amino acid with the ascorbic acid 2-glucoside. Specifically, the ascorbic acid 2-glucoside and the basic amino acid are added, at 40 to 60° C., in ion-exchanged water in which the concentration of dissolved oxygen is reduced by ultrasonic treatment, for example, and pH is adjusted to 5.22 to 5.55, thereby adjusting the amount of the basic amino acid to be added and causing reaction. After completion of the reaction, the ascorbic acid 2-glucoside basic amino acid salt can be isolated as a powder by freeze drying, and spray drying, for example.

The obtained ascorbic acid 2-glucoside basic amino acid salt powder is stable for a long period of time and is not browned, and thus is useful as a raw material for blending in cosmetics or pharmaceutical compositions at a high concentration. In case of solution, there has been a restriction to the upper limit of the concentration because it is mixed with other bases. In order to store the raw material in a liquid state, it is necessary to add a preservative, for example, to sterilize the raw material by passing through a filter and fill the raw material in a sterile container, or to store the raw material at low temperature, for example. However, by powdering the raw material, the raw material can be stably and compactly stored at room temperature without adding other components.

The ascorbic acid 2-glucoside basic amino acid salt is extremely stable without browning even in an aqueous solution as shown in Examples described later. This effect is entirely unexpected considering that ascorbic acid is browned by undergoing a Maillard reaction with normal amino acids.

Therefore, the composition containing the ascorbic acid 2-glucoside basic amino acid is useful as a browning-suppressed composition for foods, medicines, and cosmetics, for example.

The content of the ascorbic acid 2-glucoside basic amino acid salt in the composition of the present invention is preferably 1 to 40 mass %, more preferably 2 to 35 mass %, and still more preferably 5 to 30 mass % from the viewpoint of the browning suppressing effect and exhibiting the functionality of the ascorbic acid 2-glucoside basic amino acid salt as ascorbic acid.

Examples of the composition containing ascorbic acid 2-glucoside basic amino acid salt include a food composition, a pharmaceutical composition, and a cosmetic composition. Examples of the form of these compositions include a liquid composition, an emulsion composition, a powder composition, and a solid composition.

Examples of the food composition include functional foods and foods for specified health uses. Examples of the pharmaceutical composition include preparations for oral administration such as a tablet, a granule, and a syrup, preparations for transdermal administration such as a cream, an ointment, and a solution, an eye drop, and an injection preparation. Examples of the cosmetic composition include liquid cosmetics, emulsion cosmetics, powdery cosmetics, and solid cosmetics. In addition to the ascorbic acid 2-glucoside basic amino acid salt, these cosmetics preferably contain water, an oil agent, a surfactant, a moisturizing component, alcohols, polyols, an ultraviolet absorber, an ultraviolet protecting agent, various powders, various cosmetic components, an antioxidant, an antibacterial agent, and a fragrance, for example.

The cosmetic of the present invention is more preferably a cosmetic for skin from the viewpoint of sufficiently obtaining the function of the ascorbic acid 2-glucoside basic amino acid salt. In addition, the form is preferably an aqueous liquid cosmetic, an oil-in-water emulsion cosmetic, or a water-in-oil emulsion cosmetic.

pH of the composition of the present invention is preferably 5.0 to 7.5, more preferably 5.0 to 6.5, and still more preferably 5.0 to 6.0 from the viewpoint of the browning suppressing effect and the reduction in irritation when applied to the skin.

By further blending ascorbic acid or salt thereof to the composition containing the ascorbic acid 2-glucoside basic amino acid salt of the present invention, it is possible to obtain a composition having excellent usability, particularly a cosmetic.

Therefore, another embodiment of the present invention is a composition containing (a) the ascorbic acid 2-glucoside basic amino acid salt and (b) the ascorbic acid or salt thereof.

The salt of ascorbic acid may be salt of ascorbic acid and a basic substance, and examples thereof include one or more selected from ascorbic acid alkali metal salt, ascorbic acid amino acid salt, and ascorbic acid amine salt. Examples of the ascorbic acid alkali metal salt include sodium salt, potassium salt, and lithium salt of ascorbic acid. Examples of the ascorbic acid amino acid salt include glycine salt, alanine salt, valine salt, leucine salt, isoleucine salt, lysine salt, arginine salt, histidine salt, serine salt, threonine salt, cysteine salt, methionine salt, asparagine salt, glutamine salt, proline salt, phenylalanine salt, tyrosine salt, and tryptophan salt of ascorbic acid. Examples of the amine salt of ascorbic acid include ammonium salt, alkylamine salt, and alkanolamine salt of ascorbic acid. Among them, the ascorbic acid alkali metal salt is more preferable.

The constituent molar ratio of the ascorbic acid to basic substance in the ascorbic acid salt is preferably 1:1.5 to 1.5:1, and more preferably 1:1.2 to 1.2:1.

The content molar ratio (a/b) of the component (a) to the component (b) in the composition of the present invention is preferably 0.2 to 5.0, more preferably 0.3 to 3.0, and still more preferably 0.5 to 2.0 from the viewpoint of the effect of suppressing browning of the composition, the effect of exhibiting a function as the ascorbic acid, and the feeling of use such as sticky feeling.

The composition of the present invention preferably has a pH of from 5.0 to 7.5, more preferably from 5.0 to 6.5, and still more preferably from 5.0 to 6.0 from the same viewpoint as described above.

Examples of the composition containing the ascorbic acid 2-glucoside basic amino acid salt and the ascorbic acid or the salt thereof include the food composition, the pharmaceutical composition, and the cosmetic composition as described above. Examples of the form of these compositions include a liquid composition, an emulsion composition, a powder composition, and a solid composition.

Examples of the food composition include functional foods and foods for specified health uses. Examples of the pharmaceutical composition include preparations for oral administration such as a tablet, a granule, and a syrup, preparations for transdermal administration such as a cream, an ointment, and a solution, an eye drop, and an injection preparation. Examples of the cosmetic composition include liquid cosmetics, emulsion cosmetics, powdery cosmetics, and solid cosmetics. In addition to the ascorbic acid 2-glucoside basic amino acid salt and the ascorbic acid or the salt thereof, these cosmetics preferably contain water, an oil agent, a surfactant, a moisturizing component, alcohols, polyols, an ultraviolet absorber, an ultraviolet protecting agent, various powders, various cosmetic components, an antioxidant, an antibacterial agent, and a fragrance, for example.

In addition, the cosmetic of the present invention is more preferably a cosmetic for skin from the viewpoint of sufficiently obtaining the function of ascorbic acid 2-glucoside basic amino acid salt and the ascorbic acid or the salt thereof. In addition, the form is preferably an aqueous liquid cosmetic, an oil-in-water emulsion cosmetic, or a water-in-oil emulsion cosmetic.

### EXAMPLES

Next, the present invention will be described in more detail with reference to Examples. However, the present invention is not limited to these Examples.

### [Preparation of Test System]

For a reaction solution of ascorbic acid (1%) and amino acid (0.12%), the difference in degree of browning (chromaticity) based on the type of amino acid was examined for a 10 mM of phosphate citrate buffer solution pH 7.0.
Measurement wavelength: 480 nm, measurement time: 10 days after production

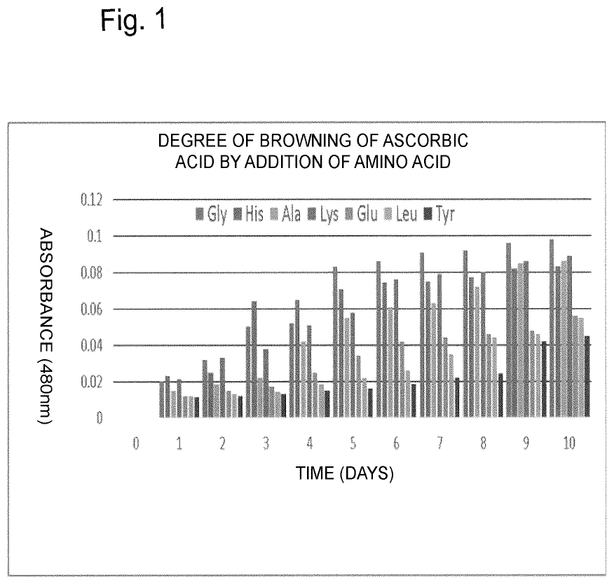

The results are shown in FIG. 1. As a result, it was suggested that the degree of browning (chromaticity) was high when the amino acid to be added to ascorbic acid was glycine, histidine, lysine, or alanine, and particularly, the degree of browning was remarkable when the glycine or the histidine was added, and the rate of degree of browning was also high.

Therefore, in the system in which the amino acid is added to ascorbic acid, browning of ascorbic acid proceeds very quickly, and it has been found that the test system in which the amino acid is added to the ascorbic acid is suitable as a screening system for the component for suppressing browning of amino acid.

### Production Example 1

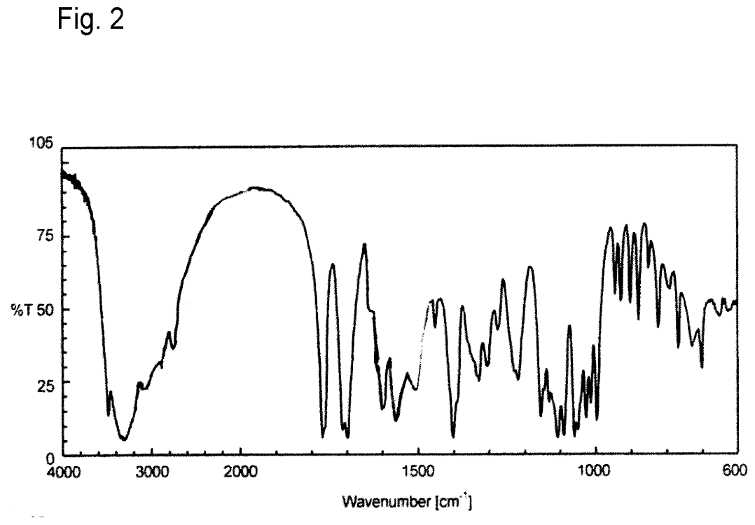

Ion-exchanged water was degassed with an ultrasonic wave having a power of 0.5 kW to 1.0 kW. Degassing was performed for 20 to 40 minutes at the time of setting 0.8 kW, and the degassing was confirmed by measuring with a dissolved oxygen concentration meter, and it was confirmed that the dissolved oxygen concentration value was 0.5 ppm or less, and the ion-exchanged water was obtained. 50 ml of this water was set to 45 to 60° C., more preferably 50° C.±0.5. 5.0 g of ascorbic acid 2-glucoside was collected, stirred and dissolved, and then 14.464 mmol of arginine was added thereto. At this time, pH is preferably 5.22 to 5.55, and more preferably 5.35. At this time, the molar concentration of arginine to be added is finely adjusted by pH. This solution was freeze-dried to obtain a powder of ascorbic acid 2-glucoside arginine salt (7.55 g, 90.03%). The FT/IR spectrum of the obtained ascorbic acid 2-glucoside arginine salt powder is shown in FIG. 2. In the spectrum result, 1720 cm⁻¹ (Non-ionic COOH, antisymmetric stretching) and a specific band of 1400 cm⁻¹ were observed, and introduction of an ionic bond of arginine was confirmed.

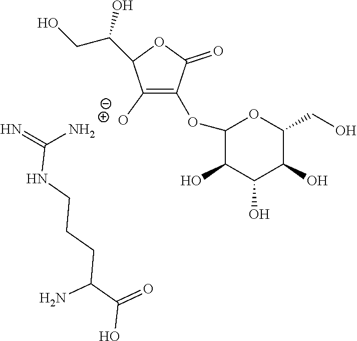 [SMILES: N=C([NH3+])NCCCC(N)C(=O)O ; O=C1OC([C@@H](O)CO)C([O-])=C1OC1O[C@H](CO)[C@@H](O)[C@H](O)[C@H]1O]

### Production Example 2

The treatment method for ion-exchanged water used for dissolution was performed in the same manner as in Production Example 1. 100 ml of this ion-exchanged water was set to 50° C.±0.5, 5.0 g of ascorbic acid 2-glucoside was added, stirred and dissolved, and then 17.97 mmol of lysine was added. pH was 5.1 to 5.6. However, it made to be pH 5.3 by finely adjusting the molar concentration. This solution was freeze-dried to obtain a powder of ascorbic acid 2-glucoside lysine salt (7.62 g, 98.42%).

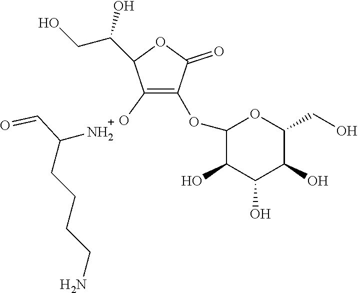 [SMILES: NCCCCC([NH3+])C=O ; O=C1OC([C@@H](O)CO)C([OH2+])=C1OC1O[C@H](CO)[C@@H](O)[C@H](O)[C@H]1O]

### Example 1

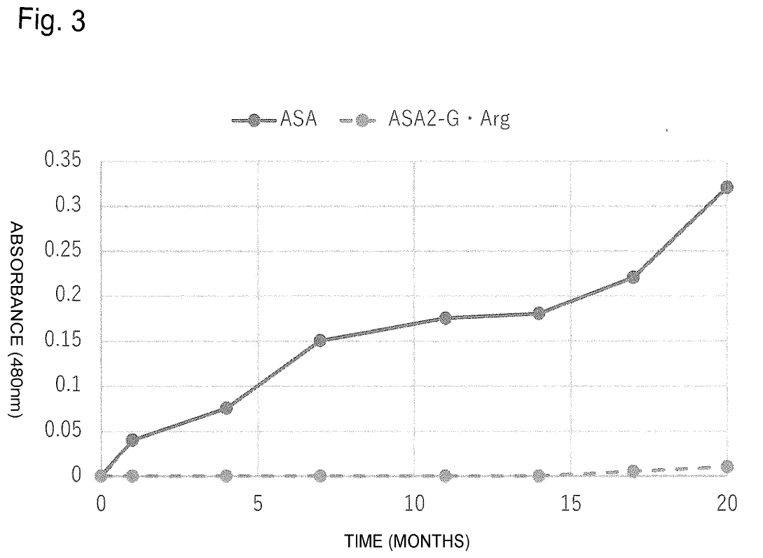

(Effect of addition of ascorbic acid 2-glucoside arginine salt on change in degree of browning (chromaticity) of ascorbic acid by addition of amino acid) 100 ml of water was taken to dissolve 1.5% of ascorbic acid (ASA), and the solution was adjusted to pH 5.0 with a 1M of sodium hydroxide solution to obtain a test solution. Ascorbic acid 2-glucoside arginine salt (ASA2-G⋅Arg) is used as a test solution by dissolving 1.5% of the product of Production Example 1 in 100 ml of water. To each test solution, 0.2% glycine was added and dissolved to prepare a solution, and the solution was stored (at room temperature) for 20 months. This test solution was collected every designated month, the absorption spectrum (wavelength: 480 nm) was measured, the degree of browning (chromaticity) was evaluated, and the results are shown in FIG. 3.

### [Evaluation]

### Appearance (Transparency)

At time 0, the initial state immediately after the preparation was visually observed. In the other designated months, a spectrophotometer (absorption spectrum: wavelength 480 nm) was used for the measurement. In the case of ascorbic acid, browning started to occur at 4 months, transparency was visually observed, and light brown color remarkably appeared at 14 months. Meanwhile, in the ASA2-G⋅Arg added system, the initial state was confirmed. In addition, the ASA 2-G⋅Arg added system visually exhibited the initial state in observation at 20 months, and the absorbance (Abs) was 0.01 in the absorption spectrum.

### Example 2

### Experiment 2: Change in Degree of Browning (Chromaticity) in Ascorbic Acid and Ascorbic Acid 2-Glucoside Lysine Salt (Production Example 2) by Addition of Amino Acid Mixture

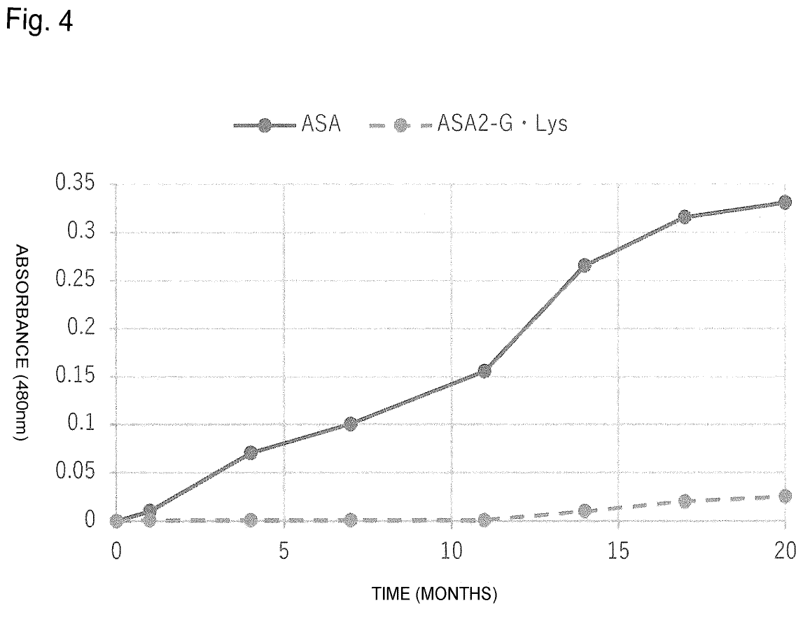

4% of ascorbic acid (ASA) is dissolved in 200 ml of water, and the solution is adjusted to pH 5.0 with a 1M sodium hydroxide solution to obtain a test solution. Ascorbic acid 2-glucoside lysine salt (ASA2-G⋅Lys) (Production Example 2) is dissolved in the same manner by 4% to prepare a test solution. An amino acid mixture (histidine 1.5%, alanine 1.28, leucine 2.6%) was added to and dissolved in each test solution, and the solution was stored (at room temperature) for 20 months. Each test solution was collected every designated month, the absorption spectrum (wavelength: 480 nm) was measured, the degree of browning (chromaticity) was evaluated, and the results are shown in FIG. 4.

### [Evaluation] Appearance (Transparency)

In this measurement, as a result of adding the amino acid mixture to the test solution, after 4 months, browning appeared in the same manner as in Example 1 with ascorbic acid (ASA), and thereafter, a browning phenomenon rapidly occurred, and a reddish brown color was exhibited at 20 months. ASA 2-G. Lys showed an extremely light reddish brown color after 20 months, and at this time, the absorbance (Abs) of the absorption spectrum was 0.017.

### Example 3

A formulation example and a production method when a serum is produced by blending 10 mass % of ascorbic acid 2-glucoside arginine salt (ASA2-G⋅Arg) are shown below.

### (1) Compounding (Unit: % by Mass)

1. Water 67.98%
2. Propanediol 17.08
3. ASA2-G. Arg 10.0%
4. Glycerin 3.9%
5. Na citrate 0.5%
6. 1,3-BG 0.30%
7. Glycosphingolipid 0.15%
8. Xanthan gum 0.12%
9. Na hyaluronate 0.02%
10. Citric acid 0.02%
11. Acetyl hydroxyproline 0.01%
12. Hydrolyzed collagen 0.001%
13. Hydrolyzed elastin 0.00001%

### (2) Production Method

Oil phase components are mixed and heated to about 80° C. Next, the remaining raw materials (water-soluble components) are mixed and heated to about 80° C. While maintaining the temperature at 80° C., the oil phase components are gradually added to the water-soluble components and emulsified while stirring with a homomixer. Finally, after the temperature is cooled to room temperature, ASA2-G⋅Ag is added, and the mixture is well mixed to be uniform, and then a product is obtained.

### Example (Comparison of Stability Between ASG and ASA2-G⋅Arg)

A serum containing 5 mass % of ASA2-G⋅Arg and ascorbic acid 2-glucoside (ASG) is prepared in the same manner as in the above Examples and used as a sample. A transparent 30 mL glass bottle with a lid was filled, and stored at room temperature under the condition of an illuminance of 30,000 lux. Absorbance at 420 nm was measured at 0, 1, 2, 3, 4, 5 and 6 months. Samples were diluted 100 fold with water at the time of measurement. Water was used as a blank.

TABLE 1

| | 0 | 1 | 2 | 3 | 4 | 5 | 6 |
|---|---|---|---|---|---|---|---|
| TEST PRODUCT | MONTHS | MONTH | MONTHS | MONTHS | MONTHS | MONTHS | MONTHS |
| LOTION | 0.00 | 0.00 | 0.00 | 0.00 | 0.01 | 0.03 | 0.05 |
| CONTAINING ASG | | | | | | | |
| LOTION | 0.00 | 0.00 | 0.00 | 0.00 | 0.00 | 0.00 | 0.00 |
| CONTAINING | | | | | | | |
| ASA2-G•Arg | | | | | | | |

In the ASG-containing serum, browning was observed over time. However, in the ASA2-G⋅Arg containing serum, browning was not observed at all by the 6 months of continued testing.

Example (mixture of ASA2-G⋅Arg and Na ascorbic acid) A serum in which ASA2-G⋅Arg and sodium ascorbate (ASNa) were mixed (the total concentration of these components was 30 mass %) was prepared, applied to the faces of 6 subjects, and questionnaire results of the feel of use (sticky feel) were obtained. The scores of five grades from good to bad were averaged.

TABLE 2

| TEST PRODUCT NUMBER | | 1 | 2 | 3 | 4 | 5 | 6 |
|---|---|---|---|---|---|---|---|
| MIXTURE | ASA2-G•Arg | 100 | 80 | 60 | 40 | 20 | 0 |
| RATIO % | ASNa | 0 | 20 | 40 | 60 | 80 | 100 |

| | | | | | | |
|---|---|---|---|---|---|---|
| SCORE AVERAGE | 3.5 | 4.4 | 4.5 | 4.7 | 4.8 | 4.6 |

In Test Product 1, there was a subject who had an impression that the Test Product 1 was sticky after application. In Test Product 2 to 6, the feel of use was almost the same. It was found that when ASNa was blended in an amount of 20% or more, usability was significantly improved. Further, in the Test Product 1 to 5, the degree of browning when stored at 40° C. was almost the same, and there was no difference.

1. A browning-suppressed composition comprising ascorbic acid 2-glucoside basic amino acid salt.

2. The composition according to claim 1, wherein the ascorbic acid 2-glucoside basic amino acid salt is one or more selected from the group consisting of ascorbic acid 2-glucoside lysine salt, ascorbic acid 2-glucoside arginine salt, ascorbic acid 2-glucoside histidine salt, and ascorbic acid 2-glucoside tryptophan salt.

3. The composition according to claim 1, wherein pH of the composition is 5.0 or more and 7.5 or less.

4. The composition according to claim 1, which is a food composition, a pharmaceutical composition, or a cosmetic composition.

5. A composition comprising
ascorbic acid 2-glucoside basic amino acid salt and
ascorbic acid or salt thereof.

6. The composition according to claim 5, wherein the ascorbic acid or the salt thereof is one or more selected from the group consisting of ascorbic acid, ascorbic acid alkali metal salt, ascorbic acid amino acid salt, and ascorbic acid amine salt.

7. The composition according to claim 5, wherein the ascorbic acid 2-glucoside basic amino acid salt is one or more selected from the group consisting of ascorbic acid 2-glucoside lysine salt, ascorbic acid 2-glucoside arginine salt, ascorbic acid 2-glucoside histidine salt, and ascorbic acid 2-glucoside tryptophan salt.

8. The composition according to claim 5, wherein pH of the composition is 5.0 or more and 7.5 or less.

9. The composition according to claim 5, which is a food composition, a pharmaceutical composition, or a cosmetic composition.

10. A powder of ascorbic acid 2-glucoside basic amino acid salt.
